(19) United States
(12) Patent Application Publication
(10) Pub. No.: US 2026/0234221 A1
(43) Pub. Date: Aug. 13, 2026

(54) HYPERIMMUNE GLOBULIN COMPOSITIONS FOR USE IN THE TREATMENT OF COVID-19

(71) Applicant: Grifols Worldwide Operations Limited, Clondalkin, Dublin (IE)

(72) Inventors: PETER VANDEBERG, DURHAM, NC (US); TODD WILLIS, DURHAM, NC (US)

(21) Appl. No.: 19/131,191

(22) PCT Filed: Nov. 17, 2023
(86) PCT No.: PCT/EP2023/082207

§ 371 Date: May 19, 2025

Related U.S. Application Data
(60) Provisional application No. 63/384,291, filed on Nov. 18, 2022.

(51) Int. Cl. C07K 16/104; C07K 16/06

(52) U.S. Cl. CPC C07K 16/104; C07K 16/06; C07K 2317/21; C07K 2317/76

(57) ABSTRACT

The disclosure relates to a pharmaceutical composition comprising human plasma-derived polyclonal, anti-SARS-CoV-2 hyperimmune globulins and to said pharmaceutical composition for use in the treatment of a SARS-CoV-2 Omicron variant infection. The disclosure also relates to a method for the preparation of a polyclonal, hyperimmune globulin composition anti-SARS-CoV-2.

### FIELD OF THE INVENTION

The present invention is related to the field of pharmaceutical products. In particular, the present invention refers to hyperimmune globulin compositions comprising human plasma-derived immunoglobulins prepared from SARS-CoV-2 convalescent plasma obtained from patients that underwent coronavirus disease (COVID-19) and who had been vaccinated from SARS-CoV-2, methods for preparing thereof, and their use in the treatment of COVID-19 in a patient in need thereof.

### BACKGROUND OF THE INVENTION

COVID-19 is a respiratory tract infection caused by a newly emergent coronavirus, severe acute respiratory syndrome coronavirus 2 (SARS-CoV-2), that was first recognized in Wuhan, China, in December 2019 (WHO Interim guidance 13 Mar. 2020). Genetic sequencing of the virus suggests that SARS-CoV-2 is a betacoronavirus closely linked to the severe acute respiratory syndrome (SARS) virus (Team NCPERE 2020). While most people with COVID-19 develop mild or uncomplicated illness, approximately 14% develop severe disease requiring hospitalization and oxygen support and 5% require admission to an intensive care unit (Team NCPERE 2020). In severe cases, COVID-19 can be complicated by acute respiratory disease syndrome (ARDS), sepsis and septic shock, multiorgan failure, including acute kidney injury and cardiac injury (Yang et al., 2020). Older age and co-morbid disease have been reported as risk factors for death, and recent multivariable analysis confirmed older age, higher Sequential Organ Failure Assessment (SOFA) score and d-dimer >1 pg/L on admission were associated with higher mortality. That study also observed median duration of viral RNA detection was 20.0 days (interquartile range [IQR] 17.0-24.0) in survivors, but SARS-CoV-2 virus was detectable until death in non-survivors. The longest observed duration of viral shedding in survivors was 37 days (Huang et al., 2020; Zhou et al., 2020). The lack of disease-directed therapeutic options for the treatment of COVID-19 has led to urgent interventions in anticipation of some potentially promising effects. Some antivirals are currently under evaluation. These include favipirivir (AVIGAN®) manufactured by Fujifilm in Japan, remdesivir manufactured by Gilead, and Kaletra® (lopinavir/ritonavir) commercially available for human immunodeficiency virus (HIV). There are also investigations of chloroquine and hydroxychloroquine as treatment modalities and potential applications for post-exposure prophylaxis according to Clinicaltrials.gov and other clinical trial registries. These and other potential therapeutic agents are described on the World Health Organization (WHO) website.

One of the approaches for the treatment of COVID-19 is the passive immunity; i.e. administering to a patient with plasma from donors that have been recovered from COVID-19 and have antibodies against this infection (hyperimmune plasma). This is known as SARS-CoV-2 convalescent human plasma, and it can be used in the treatment of COVID-19 in patients in need thereof to reduce all-cause mortality in requiring or not intensive care unit (ICU) admission patients and/or to reduce clinical severity, duration of hospital and ICU stay, dependency of oxygen and ventilator support.

Convalescent plasma has a long history of treatment of infectious diseases extending from the Spanish flu pandemic (Luke, T. C., et ai, 2006) to more recent outbreaks of severe acute respiratory syndrome (SARS) (Soo, Y. O., et ai, 2004), Middle East respiratory syndrome (MERS) (Ko, J. H., et ai, 2018) and Ebola (Mupapa, K., et ai, 1999).

There are, however, some disadvantages of convalescent plasma, including that the nature, titer and neutralizing power of the antibodies therein can vary greatly from one donor to another. In addition, there are risks associated with the volume of convalescent plasma infused (transfusion-associated circulatory overload), the need to match donor/recipient blood types, the potential for transfusion-related allergic reactions and the lack of validated pathogen reduction processes.

Thus, there is a need for new therapeutic compositions that can overcome the above disclosed drawbacks of using convalescent plasma. In particular, it would be desirable to provide a composition having higher titers that could be administered in patients with lower volumes of administration and to patients with impaired immune systems. Also, it would be desirable to have a therapeutic composition that would maintain an effective neutralizing activity for new SARS-CoV-2 variants.

The inventors of the present application have found that hyperimmune globulin compositions made from pooled plasma from donors that underwent coronavirus disease (COVID-19) and who had been vaccinated from SARS-CoV-2, can be used in the treatment of COVID-19. Indeed, this new composition surprisingly shows higher potency than compositions comprising human plasma-derived immunoglobulins prepared from SARS-CoV-2 convalescent plasma. Also, surprisingly, this hyperimmune globulin composition has activity against all variants of SARS-Cov-2 virus, including Omicron variants, although the plasma used to manufacture the composition was collected before the availability of the Omicron booster vaccine and before the Omicron variant was prevalent in the population.

### SUMMARY OF THE INVENTION

As mentioned above, the pharmaceutical hyperimmune globulin composition comprising human plasma-derived immunoglobulins prepared from SARS-CoV-2 convalescent plasma from patients that underwent coronavirus disease (COVID-19) and who had been vaccinated from SARS-CoV-2 surprisingly shows higher titers than hyperimmune globulin compositions prepared from SARS-CoV-2 convalescent plasma from patients that underwent coronavirus disease (COVID-19) but not been vaccinated from SARS-CoV-2. Moreover, unexpectedly, the hyperimmune globulin composition of the invention shows neutralization of a panel of variants, including Delta and Omicron variants even though the plasma used to manufacture the composition was collected before the availability of the Omicron booster vaccine and before the Omicron variant was prevalent in the population.

Despite the absence of antibodies raised specifically to Omicron variants, the hyperimmune globulin composition of the invention shows neutralization of a panel of variants, including Delta and Omicron variants to the same degree as neutralization of the ancestral virus strain when compared in a live virus neutralization assay.

In addition, the inventors of the present application have developed a method for preparing said hyperimmune globulin composition resulting in a composition with improved antibody titer and neutralization activity in relation to convalescent plasma-derived hyperimmune globulin compositions.

Thus, in a first aspect, the present invention refers to a pharmaceutical composition comprising human plasma-derived polyclonal, hyperimmune globulins anti-SARS-CoV-2 wherein said plasma is a convalescent plasma from a subject having recovered from SARS-CoV-2 infection and who had been vaccinated from SARS-CoV-2.

In another aspect, the invention provides a method for the manufacture of a polyclonal, hyperimmune globulin composition anti-SARS-CoV-2 comprising preparing a plasma sample from a subject who has been recovered from SARS-CoV-2 infection and who had been vaccinated from SARS-CoV-2.

As SARS-Cov-2 continues to mutate, the method of the invention will provide compositions showing effective activity against SARS-CoV-2 new variants.

In another aspect, the invention provides a method for determining the amount of the pharmaceutical composition comprising human plasma-derived polyclonal, anti-SARS-CoV-2 hyperimmune globulins of the invention to be administered to a subject in need thereof.

### DESCRIPTION OF THE FIGURES

FIG. 1 shows virus neutralization studies results. IC50 is a quantitative measure of the neutralizing activity. Titration curves A) show increased potency of composition of the invention (V02) compared to Version 01 (V01) against the ancestral virus, WA.01. Titration curves B) show Version 02 (V02) study drug neutralization of three different omicron strains.

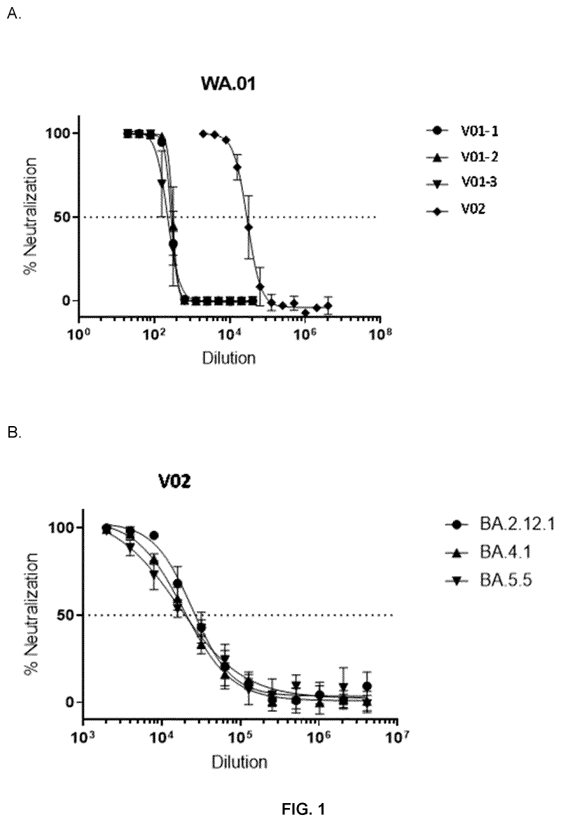

### DETAILED DESCRIPTION OF THE INVENTION

As used herein, the section headings are for organizational purposes only and are not to be construed as limiting the described subject matter in any way. Al<meal literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “Nucleic acid technology or NAT”, as used herein, refers to any amplification-based or transcription-based method for detection and quantitation of a target nucleic acid. Numerous amplification-based methods are well known and established in the art, such as PCR, its variation RT-PCR, strand displacement amplification (SDA), thermophilic SDA (tSDA), rolling circle amplification (RCA), helicase dependent amplification (HDA), or loop-mediated isothermal amplification (LAMP). Transcription-based amplification methods commonly used in the art include nucleic acid sequence-based amplification (NASBA), Ob replicase, self-sustained sequence replication or transcription-mediated amplification (TMA).

The term “convalescent plasma”, as used herein, refers to plasma collected from previously infected individuals. Thus, the term “convalescent anti-SARS-CoV-2 plasma” or “SARS-CoV-2 convalescent human plasma” as used herein refer to convalescent plasma collected from individuals previously infected with SARS-CoV-2 that have recovered from COVID-19 and that are in a convalescent noninfectious state.

The term “hyperimmune”, as used herein, refers to products or compositions comprising an elevated level of antibodies, e.g, polyclonal antibodies, to one or more specific antigens, which is obtained from plasma and/or serum.

The term “plasma-derived”, as used herein, refers to products that are made from donated human blood, from which the plasma or plasma proteins (such as immonolubulins) are separated or removed and made into proteins concentrates or fresh frozen plasma. Plasma-derived products can be made from pools of samples from multiple donors.

The terms “neutralization activity” or “IC50 neutralization titre” as used herein, are interchangeable and refer to the amount of the composition of the present invention, required for neutralizing or inhibiting 50% of infection by SARS-CoV-2.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure.

It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above. It should be understood, however, that this description, while indicating preferred embodiments of the disclosure, is given by way of illustration only, since various changes and modifications within the spirit and scope of the disclosure will become apparent to those skilled in the art.

The terminology used in the description presented herein is not intended to be interpreted in any limited or restrictive manner. Rather, the terminology is simply being utilized in conjunction with a detailed description of embodiments of the systems, methods and related components. Furthermore, embodiments may comprise several novel features, no single one of which is solely responsible for its desirable attributes or is believed to be essential to practicing the embodiments herein described.

In a first aspect, the present invention refers to a pharmaceutical composition comprising human plasma-derived polyclonal, hyperimmune globulins anti-SARS-CoV-2 wherein said plasma is a convalescent plasma from a subject having recovered from SARS-CoV-2 infection and who had been vaccinated from SARS-CoV-2.

It can be appreciated that the antibody response in donors can be used to determine whether they have convalesced from natural SARS-CoV-2 infection, been vaccinated or both (hybrid). Convalescence from the SARS-CoV-2 virus will prompt an immune response that produces antibodies to both the spike protein (anti-S) and to the nucleocapsid protein (anti-N) of the virus (Jones, Jefferson M., et al. “Estimates of SARS-CoV-2 seroprevalence and incidence of primary SARS-CoV-2 infections among blood donors, by COVID-19 vaccination status-United States, April 2021-September 2022.” Morbidity and Mortality Weekly Report 72.22 (2023): 601.

Furthermore, the levels of anti-S and neutralization in hybrid donors is ten-fold or more higher than those produced by convalescence alone (Di Germanio, Clara, et al. “Vaccination of COVID-19 convalescent plasma donors increases binding and neutralizing antibodies against SARS-CoV-2 variants.” Transfusion 62.3 (2022): 563-569.). Thus, hybrid donors will have a characteristic response of high anti-S levels and the presence of some anti-N. Hence according to this information it can be determined whether plasma is a convalescent plasma from a subject having recovered from SARS-CoV-2-infection and who had been vaccinated from SARS-CoV-2.

In a particular embodiment, said subject has recovered from non-omicron infections.

The inventors have demonstrated by performing neutralizing activity tests, that Omicron variants show no significant resistance to neutralization when the composition of the invention is being tested. As shown in the Examples accompanying the present invention, despite the absence of antibodies raised specifically to Omicron variants, the hyperimmune globulin composition of the invention shows neutralization of a panel of variants, including Delta and Omicron variants to the same degree as neutralization of the ancestral virus strain when compared in a live virus neutralization assay (Example 3).

In another particular embodiment, the hyperimmune globulin composition of the invention lacks a significant reduction in potency to an omicron BA4 and/or BA5 variant. Thus, an advantage of the hyperimmune globulin composition of the invention is that it could be used for the treatment of new variants of SARS-Cov-2 virus. This is an unexpected result since the plasma used to manufacture the composition of the invention was collected before the availability of the Omicron booster vaccine and before the Omicron variant was prevalent in the population. Surprisingly, the composition of the invention showed neutralization of a panel of variants, including Delta and Omicron variants.

The hyperimmune globulin composition of the invention is particularly beneficial for patients with impaired immune systems.

In a particular embodiment, the composition of the invention comprises human plasma-derived immunoglobulin G (IgG) with a purity of at least 97% of the total protein content. In some embodiments, said composition comprises human plasma-derived immunoglobulin G (IgG) with a purity of at least 98%, at least 99%, at least 99.5%, at least 99.8% or at least 99.9% of the total protein content. In some embodiments said composition comprises human plasma-derived immunoglobulin G (IgG) with a purity of about 100%.

In another particular embodiment, the composition of the invention can have a SARS-CoV-2 antibody titre between 250,000 and 2,500,000. In some embodiments, the SARS-CoV-2 antibody titre of the composition of the present invention is between 500,000 and 2,000,000. In other embodiments, said SARS-CoV-2 antibody titre is between 750,000 and 1,500,000. In other embodiments, said SARS-CoV-2 antibody titre is between 1,000,000 and 1,250,000. In other embodiments, said SARS-CoV-2 antibody titre is between 1,100,000 and 1,200,000. In some embodiments the SARS-CoV-2 antibody titre is greater than 500,000, preferably greater than 750,000, preferably greater than 1,000,000, preferably greater than 1,250,000, preferably greater than 1,500,000, preferably greater than 2,000,000.

In some embodiments of the present invention, the SARS-CoV-2 antibody titre of composition of the invention is increased by at least 2-fold with respect to the SARS-CoV-2 antibody titre in the pooled plasma from which said composition is prepared. In other embodiments, said SARS-CoV-2 antibody titre is increased by at least 5-fold, preferably by at least 10-fold, preferably by at least 15-fold, preferably by at least 25-fold, preferably by at least 30-fold, with respect to the SARS-CoV-2 antibody titre in the pooled plasma from which said composition is prepared.

The SARS-CoV-2 antibody titre of the present composition can be determined using, for example, the human Anti-SARS-CoV-2 Virus Spike 1 (S1) IgG assay from Alpha Diagnostics Ltd. (Switzerland). However, other assays known by the skilled person can also be used.

The SARS-CoV-2 antibody titre of the composition of the invention can also be normalized per mg/ml of IgG. Thus, in some embodiments the SARS-CoV-2 antibody titre of the composition of the present invention is between 2,500 and 25,000 per mg/mL of IgG. In other embodiments, said SARS-CoV-2 antibody titre is between 3,000 and 22,000 per mg/ml of IgG. In other embodiments, said SARS-CoV-2 antibody titre is between 3,500 and 20,000 per mg/ml of IgG. In other embodiments, said SARS-CoV-2 antibody titre is between 4,000 and 19,000 per mg/ml of IgG. In other embodiments, said SARS-CoV-2 antibody titre is between 4,500 and 18,000 per mg/ml of IgG. In other embodiments, said SARS-CoV-2 antibody titre is between 4,850 and 17,000 per mg/ml of IgG. In some embodiments, said SARS-CoV-2 antibody titre is greater than 3,000, preferably greater than 5,000, preferably greater than 7,500, preferably greater than 10,000, preferably greater than 15,000, preferably greater than 20,000 per mg/ml of IgG. The composition of the present invention can have a SARS-CoV-2 neutralization activity (IC50 neutralization titer) between 7,500 and 75,000. In some embodiments, the SARS-CoV-2 neutralization activity is between 10,000 and 62,500. In more preferred embodiments, said neutralization activity is between 12,500 and 50,000. In yet more preferred embodiments, said neutralization activity is between 15,000 and 36250. In more preferred embodiments, said neutralization activity is between 20,000 and 25,000. In some embodiments, the SARS-CoV-2 neutralization activity is greater than 7,500, preferably greater than 10,000, preferably greater than 15,000, preferably greater than 20,000, preferably greater than 25,000, preferably greater than 37,500, preferably greater than 50,000.

In some embodiments of the present invention, the SARS-CoV-2 neutralization activity of the hyperimmune globulin composition is increased by at least 2-fold with respect to the SARS-CoV-2 neutralization activity in the pooled plasma from which said composition is prepared. In other embodiments, said SARS-CoV-2 neutralization activity is increased by at least 5-fold, preferably by at least 7-fold, preferably by at least 10-fold, preferably by 10 to 15-fold, with respect to the SARS-CoV-2 neutralization activity in the pooled plasma from which said composition is prepared.

The SARS-CoV-2 neutralization activity (IC50 neutralization titre) of the present hyperimmune globulin composition can be determined using, for example, an immunofluorescence-based neutralization assay in which inhibition of infection of cultured eukaryotic cells, such as Vero (CCL-81) cells by SARS-CoV-2, is tested. However, the skilled person knows other assays that can be used to determine the SARS-CoV-2 neutralization activity (IC50) of the present composition.

The SARS-CoV-2 neutralization activity (IC50 neutralization titer) of composition of the present invention can also be normalized per mg/ml of IgG. Thus, in some embodiments the SARS-CoV-2 neutralization activity of the composition of the present invention is between 75 and 1125 per mg/ml of IgG. In other embodiments, said SARS-CoV-2 neutralization activity is between 90 and 600 per mg/ml_of IgG. In more preferred embodiments, said SARS-CoV-2 neutralization activity is between 100 and 500 per mg/ml_of IgG. In yet more preferred embodiments, said SARS-CoV-2 neutralization activity is between 110 and 475 per mg/ml_of IgG. In even more preferred embodiments, said SARS-CoV-2 neutralization activity is between 120 and 440 per mg/ml_of IgG. In some embodiments, the SARS-CoV-2 neutralization activity is greater than 75, preferably greater than 100, preferably greater than 125, preferably greater than 250, preferably greater than 375, preferably greater than 500, preferably greater than 625, per mg/ml_of IgG.

The composition of the invention comprising human plasma-derived polyclonal, hyperimmune globulins anti-SARS-CoV-2 can be defined by any of the above antibody titre and/or neutralization activity.

In some embodiments, the IgG content of the composition of the present invention is between 5% and 20% (w/v). In more preferred embodiments, the human plasma-derived IgG content is between 7% and 15% (w/v). In more preferred embodiments, the human plasma-derived IgG content is between 9% and 11% (w/V). In more preferred embodiments, the human plasma-derived IgG content is around 10% (w/v).

In some embodiments, at least 90% of IgG of the hyperimmune globulin composition of the present invention is present as monomers and dimers. In more preferred embodiments, at least 95% of the human plasma-derived IgG is present as monomers and dimers. In more preferred embodiments, at least 98% of the human plasma-derived IgG is present as monomers and dimers. In more preferred embodiments, at least 99% of the human plasma-derived IgG is present as monomers and dimers. In more preferred embodiments, at least 99.8% of the human plasma-derived IgG is present as monomers and dimers.

The hyperimmune globulin composition of the present invention is a highly purified IgG composition, but it may comprise residual amounts of other immunoglobulins, such as immunoglobulin A (IgA) or immunoglobulin M (IgM). In some embodiments, the content of IgA in said composition is equal or lower than 0.04 mg/ml. In more preferred embodiments, the content of IgA in said composition is equal or lower than 0.038 mg/ml. In some embodiments, the content of IgM in said composition is equal or lower than 0.01 mg/ml.

The hyperimmune globulin composition of the present invention can be used in the treatment of coronavirus disease 2019 (COVID-19) in a patient in need thereof. As indicated before, in a preferred embodiment the composition of the invention can be used in the treatment of a SARS-CoV-2 Omicron variant infection. More particularly, the composition of the invention can be used in the treatment of SARS-CoV-2 Omicron BA4 and/or BA5 variant infection.

The skilled person knows the preferred dosage regimes and administration route for the treatment of COVID-19 with the hyperimmune globulin composition of the present invention. In a preferred embodiment, intravenous route is the preferred administration route to be used for the hyperimmune globulin composition of the present invention.

In another aspect, the invention provides a method for the manufacture of a polyclonal, hyperimmune globulin composition anti-SARS-CoV-2 comprising preparing a hyperimmune globulin composition according to the present invention from a plasma sample from a subject who has been recovered from SARS-CoV-2 infection and who had been vaccinated from SARS-CoV-2. Preferably, said plasma sample is from a subject that had recovered form a non-Omicron variant SARS-CoV-2 infection and who had been vaccinated from a non-Omicron SARS-CoV-2 variant. In a preferred embodiment, said plasma is a pool of plasma samples from at least two donors that have been recovered from SARS-CoV-2 infection and who had been vaccinated from SARS-CoV-2. In a more preferred embodiment, said plasma is a pool of plasma samples from at least ten donors that have been recovered from SARS-CoV-2 infection and who had been vaccinated from SARS-CoV-2. In an even more preferred embodiment, said plasma is a pool of plasma samples from at least one hundred donors that have been recovered from SARS-CoV-2 infection and who had been vaccinated from SARS-CoV-2. In a more preferred embodiment, said plasma is a pool of plasma samples from at least two hundred donors that have been recovered from SARS-CoV-2 infection and who had been vaccinated from SARS-CoV-2.

As shown in the Examples, live virus neutralization studies show activity of the anti-SARS-CoV-2 hyperimmune composition of the invention against a wide variety of new SARS-CoV-2 variants (Example 3). Thus, the method of the invention provides compositions showing effective activity against SARS-CoV-2 new variants. More particularly, the method of the invention provides hyperimmune globulin compositions showing effective activity against new Omicron variants, including BA4 and/or BA5 variants.

Said method includes processing steps that have been validated for virus clearance and blood typing, reducing the risks of inadvertently transferring known infectious agents or triggering transfusion reactions. Thus, another advantage of the hyperimmune globulin composition of the invention over convalescent plasma is the pathogen clearance capability built into the processing. Both, convalescent plasma for transfusion and for manufacturing require the testing of common viral agents such as human immunodeficiency virus (HIV) and hepatitis B virus. However, in the event that novel viral contaminants are present, the method described herein to manufacture the hyperimmune globulin composition of the invention includes steps validated to remove or inactivate any viral pathogens.

The method of preparing a pharmaceutical composition of the invention can be prepared from plasma using well known plasma processing techniques.

For example, the well-known caprylate/chromatography purification method can be used. Here Cohn Fractionation or Alcohol Fractionation is utilized to produce an immunoglobulin enriched paste, which is then re-solubilized and subject to the caprylate/chromatography purification process as described.

However, as way of example a protocol for the preparation of the pharmaceutical composition of the invention is provided below and in the accompanying examples.

The method of the invention, as described herein, comprises, from a starting solution comprising anti-SARS-CoV-2 antibodies, the following sequential steps a) through e) of: a) adjusting the pH of the starting solution to be within a range of from about 3.8 to about 4.5 to form an intermediate solution comprising dissolved antibodies, b) adding a source of caprylate ions to the intermediate solution of step a) and adjusting the pH of the intermediate solution to be within a range of from about 5.0 to about 5.2 to form a precipitate and a supernatant solution comprising dissolved antibodies, c) incubating the supernatant solution under conditions of time, temperature and caprylate ion concentration to inactivate substantially all viruses, d) contacting the supernatant solution with at least one ion exchange resin under conditions that allow binding of at least some of the other substances including IgA or IgM to the resin while not allowing binding of the antibodies including IgG to the resin, and e) collecting the IgG antibodies, wherein the starting solution is a SARS-CoV-2 human plasma collected from a subject who has been recovered from SARS-CoV-2 infection and who had been vaccinated from SARS-CoV-2. In a preferred embodiment, the collected plasma is a pool of plasma samples from at least two donors that have been recovered from SARS-CoV-2 infection and who had been vaccinated from SARS-CoV-2. In a more preferred embodiment, said plasma is a pool of plasma samples from at least ten donors that have been recovered from SARS-CoV-2 infection and who had been vaccinated from SARS-CoV-2. In an even more preferred embodiment, said plasma is a pool of plasma samples from at least one hundred donors that have been recovered from SARS-CoV-2 infection and who had been vaccinated from SARS-CoV-2. In a more preferred embodiment, said plasma is a pool of plasma samples from at least two hundred donors that have been recovered from SARS-CoV-2 infection and who had been vaccinated from SARS-CoV-2.

In some preferred embodiments, the method for preparing the hyperimmune globulin composition of the present invention further comprises a non sequential step f) of eluting IgA or IgM from the ion exchange resin column.

In other embodiments, the method for preparing the hyperimmune globulin composition as described herein is as disclosed in document U.S. Pat. No. 6,307,028, which is incorporated by reference herein.

In some embodiments, said SARS-CoV-2 human plasma is tested negative for at least one of blood-borne pathogens and human leukocyte antigen (HLA) antibody. Any known method for testing blood-borne pathogens can be used. Similarly, any known method for testing the presence of human leukocyte antigen (HLA) antibodies can be used.

In other embodiments, said SARS-CoV-2 human plasma is tested for blood type.

In some embodiments, the plasma pool SARS-CoV-2 antibody titre is between 3,000 and 13,000, preferably between 4,000 and 12,000, more preferably between 5,000 and 11,000. In some embodiments the SARS-CoV-2 antibody titre is greater than 2,000, preferably greater than 4,000, preferably greater than 5,000, preferably greater than 6,000.

In some embodiments, the plasma pool SARS-CoV-2 neutralization activity is between 300 and 60,000, preferably between 500 and 50,000, more preferably between 1,000 and 40,000, more preferably between 2,000 and 30,000.

The present invention refers to a method for obtaining hyperimmune human plasma from a donor for use in the treatment of coronavirus disease 2019 (COVID-19), wherein said donor has a laboratory confirmed diagnosis of COVID-19, said donor is in a convalescent noninfectious state and has been vaccinated from SARS-CoV-2. In a preferred embodiment, said donor has recovered from a non-omicron variant infection. In another preferred embodiment, said donor has not been vaccinated from a non-omicron SARS-CoV-2 variant.

In some embodiments, said donor is symptomatic or asymptomatic for COVID-19.

In some embodiments, symptoms of COVID-19 are one or more of fever, tiredness, dry cough, ache, pain, nasal congestion, runny nose, sore throat, or diarrhea.

In some embodiments, said donor is tested positive or negative for COVID-19 as determined by any nucleic acid technology (NAT) test and/or by any serology test for detecting antibodies anti-SARS-CoV-2.

In some embodiments, if said donor is asymptomatic and is tested positive for antibodies anti-SARS-CoV-2 but negative in the NAT test, is immediately eligible for plasma donation. In some embodiments, if said donor is asymptomatic and is only tested positive for the NAT test, or is positive for the NAT test with a subsequent positive for antibodies anti-SARS-CoV-2, said donor is eligible for plasma donation 28 days after collecting the sample for the NAT test.

In some embodiments, if said donor is asymptomatic and is only positive for anti-SARS-CoV-2 antibodies, said donor is eligible for plasma donation after 7 days after the serology test.

In some embodiments, if said donor is asymptomatic and is positive for the NAT test with a subsequent negative for the NAT test, said donor is eligible for plasma donation 14 days after the negative NAT test.

In some embodiments, if said donor is symptomatic and is only positive for the NAT test, or is positive for the NAT test with a subsequent positive for anti-SARS-CoV-2 antibodies, or is only positive for anti-SARS-CoV-2 antibodies, or is positive for the NAT test with a subsequent negative for anti-SARS-CoV-2 antibodies, said donor is eligible for plasma donation 28 days after cessation of all symptoms.

In some embodiments, if said donor is symptomatic and is positive for the NAT test with a subsequent positive NAT test, said donor is eligible for plasma donation 28 days after cessation of all symptoms or 28 days after the second NAT test, whichever is later.

In some embodiments, if said donor is symptomatic and is positive for the NAT test with a subsequent negative for the NAT test, said donor is eligible for plasma donation 14 days after cessation of all symptoms.

In some embodiments, said plasma is screened at least for blood-borne pathogens and blood type.

In some embodiments, said plasma is frozen after collection.

In another aspect, the invention provides a method determining the amount of the pharmaceutical composition comprising human plasma-derived polyclonal, anti-SARS-CoV-2 hyperimmune globulins of the invention to be administered to a subject in need thereof, the method comprising:
a. determining the dominant variant of SARS-CoV-2; and,
b. calculating the amount of the pharmaceutical composition according to the genotype of the dominant variant.

An embodiment of the method of the invention is wherein step (b) comprises (i) determining the variant neutralization capacity of the pharmaceutical composition for the genotype of the dominant variant, (ii) expressing the neutralization capacity in dosage unit per liter of the pharmaceutical composition, and (iii) calculating the amount of the pharmaceutical composition to be administered to the subject in need thereof.

The present inventors have determined that the amount of the pharmaceutical composition to be administered to a subject can vary according to the dominant variant of SARS-CoV-2 responsible for the infection.

As can be seen in the accompanying Examples, where the dominant variant of SARS-CoV-2 is Delta or Omicron class of variant then the amount of neutralising units to that infection in the pharmaceutical composition of the invention varies. Hence the amount to be administered to the subject should be varied so as to provide an effective therapeutic quantity for that subject.

For example, if the effective therapeutic quantity of the pharmaceutical composition to be administered to a subject is around 340, then more of the pharmaceutical composition should be administered to a patient suffering from SARS-CoV-2 infection caused by a dominant omicron (XBB.1.5) variant compared to the quantity to be administered where the SARS-CoV-2 infection is caused by a dominant omicron (BA5.5) variant of SARS-CoV-2.

Further information on the correlating of neutralising units for specific variants of SARS-CoV-2 can be seen in the accompanying Examples, particularly Table 4. It can be appreciated that the scope of the present invention should not be limited to just those variants presented in Table 4. The amount of pharmaceutical composition to be administered to a subject can be determined by the principles disclosed herein for any such variant.

An embodiment of this method of the invention is wherein step (a) comprises measuring the frequency of SARS-CoV-2 variants in a subject to be treated and/or the environmental profile of SARS-CoV-2 variants for that subject.

Methods of determining the frequency of SARS-CoV-2 variants and the environmental profile of SARS-CoV-2 variants are known in the art and can be readily used by the skilled person.

For example, sequencing of variants is often done by methods referred to as Next Generation Sequencing (NGS). An example of commercial reagents used for sequencing is the Illumina COVIDSeq Test which is a diagnostic test performed on commercially available instruments (Illumina, San Diego, CA, www.illumina.com). Reference: Lowry, Kym, Michelle J. Bauer, Cameron Buckley, Claire Wang, Amanda Bordin, Steven Badman, Patrick NA Harris, Ian Mackay, and David Whiley. “Evaluation of Illumina® COVIDSeq™ as a Tool for Omicron SARS-CoV-2 Characterisation.” Journal of virological methods (2023): 114827.

SARS-CoV-2 sequences are registered through the Global Initiative on Sharing All Influenza Data (GISAID, www.gisaid.org) and naming of variants is standardized through the PANGO network (Phylogenetic Assignment of Named Global Outbreak LINeages) and are registered at https://cov-lineages.org

The standardized nomenclature used by PANGO is described in the following publication Rambaut, Andrew, Edward C. Holmes, Áine O'Toole, Verity Hill, John T. McCrone, Christopher Ruis, Louis du Plessis, and Oliver G. Pybus. “A dynamic nomenclature proposal for SARS-CoV-2 lineages to assist genomic epidemiology.” Nature microbiology 5, no. 11 (2020): 1403-1407.

By “environmental profile” we mean determining the frequency of different variants in the environment of the subject to be administered with the pharmaceutical composition of the invention.

Methods of determining the environmental profile of viral variants, including SARS-CoV-2 variants, are known in the art. For example, samples from wastewater, atmosphere, surfaces and other such viral reservoirs, can be collected and the profile of SARS-CoV-2 variants determined according to known analytical techniques.

It is preferred that the “environmental profile” is determined from wastewater. The analysis of wastewater to determine the environmental profile of SARS-CoV-2 variants is known in the art. For example Smith et al (2023) American Society of Micobiology, vol 14, no. 1 discloses methods which can be used by the skilled person to determine the environmental profile of SARS-CoV-2 variants for the subject to be treated.

Hereinafter, the present invention is described in more detail with reference to illustrative examples, which does not constitute a limitation of the present invention.

### EXAMPLES

### Example 1: Selection of Plasma Donors for Collection of SARS-CoV-2 Convalescent Plasma Plus Vaccinated (Hybrid Plasma)

For the selection of plasma donors for obtaining SARS-CoV-2 convalescent plus vaccinated plasma for use in the production of the composition of the present invention, donors are individuals having recovered from SARS-CoV-2 infection and who has been vaccinated from SARS-CoV-2. All plasma samples were collected prior to October 2021, well before the widespread appearance of Omicron variants.

In brief, individuals in good health who have been approved through the pre-screening process are allowed to proceed to the donation center for final evaluation and donation. This pre-screening process assured that only individuals who have recovered from their illness or were exposed to the disease agent but remained asymptomatic, would qualify to come into the center and potentially donate. Moreover, individuals must had been vaccinated.

Thus, only individuals that had a laboratory evidence of COVID-19 infection, either through nucleic acid amplification testing (NAT), positive antigen test, or by SARS-CoV-2 antibody test prior to enrollment and were then in a convalescent noninfectious state may be safely processed within the donor center. Symptomatic donors had to have complete resolution of symptoms at least 14 days before the donation if they were negative by a follow-up NAT, or 28 days if they had no follow-up test. Similarly, asymptomatic donors who were positive by NAT or antigen tests were required to wait 14 days after the initial test if they had a follow-up negative NAT, but had to wait 28 days after the initial test if they had no follow-up test. Asymptomatic donors who were only tested by an anti-SARS-CoV-2 antibody test were required to wait seven days prior to donation, but could donate immediately if they also had a negative NAT.

Also, donors were tested for vaccination status. For this, a test was performed in order to distinguish convalescent (anti-N and anti-S) and vaccination (anti-S).

Donors also had to be negative for human leukocyte antigen (HLA) antibodies.

Once the donor has been selected, plasma is collected by plasmapheresis.

Each plasma unit must meet requirements for source plasma for manufacturing as defined by regulations including screening against a variety of infectious agents. Additionally, each unit was tested to confirm it was negative for SARS-CoV-2 virus and positive for anti-SARS-CoV-2 antibodies.

Each plasma sample was also tested to be negative for human leukocyte antigen (HLA) antibodies and blood typed. Then, plasma pools were modeled to maintain consistent distribution with the overall donor ABO blood type distribution to maintain consistent batch to batch levels of anti-A and anti-B.

### Example 2: Manufacture of a Liquid Therapeutic Hyperimmune Globulin Composition from SARS-CoV-2 Convalescent and Vaccinated Plasma

The plasma pools obtained as described in the Example 1 were then processed following the same steps as the Gamunex-C caprylate/chromatography process (Lebing, W., et al, 2003, U.S. Pat. No. 6,307,028, each incorporated by reference herein), which included multiple steps validated for the removal and/or inactivation of viruses (Gamunex-C [Immune Globulin Injection (Human) 10% Caprylate/Chromatography Purifiedj-Package Insert. 2020). The resulting product was a highly purified IgG solution formulated at around 10% protein content with glycine at a pH of about 4.2.

### Example 3. Characterization of SARS-CoV-2 Product

The composition of the present invention was characterized to assess the recovery of anti-SARS-CoV-2 specific antibodies. Thus, the composition of the invention was tested with an IgG specific Enzyme-linked immunosorbent assay (ELISA) and a neutralizing antibody assay.

Characterization also included prior routine batch testing to characterize the product and ascertain that it is suitable for use. This characterization included analyses for glycine, pH, protein concentration, osmolality, composition by electrophoresis, and molecular weight profiling by size exclusion chromatography. Analyses were also performed for sodium caprylate, residual IgA and IgM, prekallikrein activator (PKA), factor Xa, anti-A, anti-B, and anti-D. In addition, compendial tests for sterility and pyrogenic substances were performed on all batches.

These tests showed that the tested batches were within the batch standards for purity, formulation, molecular profile and purity described for other immune globulin products manufactured with the caprylate/chromatography process, such as Gamunex-C. The batches also passed USP pyrogen and sterility tests.

The tests showed that between 97% and 100% of the protein content was IgG. In addition, the IgG was present almost entirely as monomers and dimers with aggregates and fragments below the limits of detection. A process impurity (sodium caprylate) and plasma protein impurities were found at very low concentrations in the final product, well under the batch requirements.

The amounts of residual IgA and IgM were also below the batch requirements (less than 0.13 mg/ml and less than 0.030 mg/ml, respectively) and the concentrations known for the Gamunex-C product.

IgM has been identified as a primary source of anti-A and anti-B intravascular hemolytic activity (Flegel, W. A., 2015). The composition of the present invention was shown to contain less than 0.01 mg/ml, which greatly reduces the danger of this adverse event. In contrast, when patients are treated with convalescent plasma, they must be matched by donor blood type to reduce the chances of hemolysis.

Similarly, removal of IgA provides a potential therapeutic advantage for hyperimmune intravenous immune globulin (hIVIG) products over convalescent plasma in patients who are IgA deficient and may have been previously treated with blood products and formed antibodies to IgA. The composition of the present invention was shown to contain less than 0.04 mg/ml of IgA.

### Anti-SARS-CoV-2 ELISA

Anti-SARS-CoV-2 IgG titers were determined using Human Anti-SARS-CoV-2 Virus Spike 1 (S1) IgG assay from Alpha Diagnostic. hIVIG batches are tested using multiple serial dilutions and a curve constructed by plotting the log of the optical density as a function of the log of the dilution. The titer was defined as the dilution at which this curve is equal to the low kit standard.

Results demonstrated that ELISA activity (ELISA titer, 1:X) increased up to almost 10-fold, when processing the pooled plasma into the final product. The IgG concentration was also increased more than 10-fold from the pooled plasma to the final product.

### Anti-SARS-CoV-2 Neutralizing Antibody Assay

The hIVIG products were also tested for anti-SARS-CoV-2 antibodies using an immunofluorescence-based neutralization assay performed at the National Institutes of Health Integrated Research Facility, Frederick, MD. This assay quantifies the anti-SARS-CoV-2 neutralization titer by using a dilution series of test material to test for inhibition of infection of cultured Vero (CCL-81) cells by SARS-CoV-2 (Washington isolate, CDC). Potency was assessed using a cell-based immunosorbent assay to quantify infection by detecting the SARS-Cov-2 nucleoprotein using a specific antibody raised against the SARS-CoV-1 nucleoprotein.

The secondary detection antibody was conjugated to a fluorophore which allows quantification of individual infected cells on a high throughput optical imaging system. A minimum of 16,000 cells were counted per sample dilution across four wells-two each in duplicate plates. Data are reported based on a 4-parameter regression curve (using a constrained fit) as a 50% neutralization titer (IC50). When tested along with a reference standard, the ratio of IC50 of samples to the reference standard can be used to establish test results in units, for example, AU/mL or IU/mL.

TABLE 1
Neutralization IC50 titers of convalescent plasma-derived hyperimmune composition
vs convalescent plus vaccinated (hybrid) plasma-derived hyperimmune composition.

| | Ancestral | | | | | | | |
|---|---|---|---|---|---|---|---|---|
| | strain | Delta | Omicron | Omicron | Omicron | Omicron | Omicron | Omicron |
| Variant | (WA.01) | (B.1.617.2) | (BA.1) | (BA.1.1) | (BA.2) | (BA.2.12.1) | (BA.4.1) | (BA.5.5) |
| Approximate | N/A | Aug. 18, | Jan. 4, | Jan. 31, | Mar. 28, | Jun. 1, | Jul. 1, | Jun. 5, |
| Date of | | 2021 | 2022 | 2022 | 2022 | 2022 | 2022 | 2022 |
| Variant | | | | | | | | |
| Peak¹ | | | | | | | | |
| Test Date | Jun. 7, | Jun. 7, | Jun. 7, | Jun. 7, | Jun. 7, | Oct. 11, | Oct. 11, | Oct. 11, |
| | 2022 | 2022 | 2022 | 2022 | 2022 | 2022 | 2022 | 2022 |
| Product - No. | Neutralization IC50 Titers (1:X) | | | | | | | |
| V01 - 1 | 310 | 309 | 173 | 140 | 239 | 307 | 213 | 218 |
| V01 - 2 | 227 | 273 | 170 | 125 | 160 | 239 | 244 | 214 |
| V01 - 3 | 248 | 299 | 198 | 147 | 175 | 311 | 317 | 277 |
| V01 - 4 | 268 | 299 | 184 | 101 | 125 | 185 | 191 | 176 |
| V01 - 5 | 205 | 254 | 153 | 101 | 135 | 170 | 139 | 201 |
| V01 - 6 | 242 | 251 | 225 | 154 | 277 | 252 | 224 | 257 |
| V01 - 7 | 200 | 256 | 155 | 115 | 174 | 214 | 214 | 181 |
| V01 - 8 | 275 | 358 | 208 | 137 | 268 | 269 | 243 | 232 |
| V01 - 9 | 214 | 286 | 202 | 138 | 215 | 248 | 211 | 214 |
| V01 - 10 | 231 | 278 | 250 | 158 | 280 | 297 | 291 | 300 |
| V01 - 11 | 301 | 423 | 415 | 197 | 439 | 421 | 452 | 452 |
| V01 - 12 | 975 | 2872 | 404 | 191 | 371 | 452 | 424 | 339 |
| V01 - 13 | 865 | 3076 | 242 | 176 | 393 | 360 | 299 | 289 |
| V01 - 14 | 1054 | 3208 | 277 | 157 | 464 | 350 | 345 | 342 |
| V01 - 15 | 321 | 405 | 328 | 217 | 596 | 380 | 395 | 332 |
| V01 - | 396 ± 299 | 856 ± 1139 | 239 ± 84 | 150 ± 34 | 287 ± 138 | 297 ± 83 | 280 ± 91 | 268 ± 76 |
| Average ± STD | | | | | | | | |
| V02 - 1 | 55,254² | N/T | N/T | N/T | N/T | 26,792 | 20,703 | 16,261³ |
| Fold | 72 | N/A | N/A | N/A | N/A | 90 | 74 | 74 |
| Increase | | | | | | | | |
| (V02/V01_{avg}) | | | | | | | | |

¹Estimated based on graphical displays of PANGO entries at https://cov-lineages.org/
²Average of three tests (N = 3)
³Average of 5 tests (N = 5)
V01: convalescent plasma composition;
V02: convalescent + vaccinated (hybrid) composition

Live virus neutralization studies show similar IC50 values across Delta and Omicron variants with all V01 (hyperimmune globulin composition from convalescent plasma) batches tested. Results showed high neutralization activity of V02 (hyperimmune globulin composition from convalescent plus vaccinated plasma) with WA.01 (ancestral strain) and Omicron variants. Titers observed with V02 were 70 to 90-fold higher compared to V01.

The results showed that antibody neutralizing activity (IC50) was increased more than 10-fold from the plasma pool to the final product. This increase in neutralizing activity indicates that patients treated with human hyperimmune globulin compositions (hIVIG) compared to an equivalent volume of convalescent plasma would receive higher neutralizing activity. Alternatively, patients treated with hIVIG could receive a smaller treatment volume compared to treatment with convalescent plasma and potentially decrease the chances for transfusion-associated circulatory overload.

TABLE 2
SARS-CoV-2 infectivity neutralization by hyperimmune
globulin plasma-derived composition of the
invention compared to pooled plasma.

| | | | FOLD INCREASE |
|---|---|---|---|
| | | INVENTION | (POOL to |
| TEST | POOL | (hIVIG) | INVENTION) |
| Neutralizing | 10,639 | 159,086 | 15.0 |
| Activity (IU/mL) | | | |
| Neutralizing | 4057 | 41,129 | 10.1 |
| Activity (AU/mL) | | | |

An advantage of using SARS-CoV-2 convalescent human plasma to manufacture the hIVIG composition of the present invention (compared to direct administration of plasma from individuals or administration of a monoclonal antibody) is the diversity of antibodies obtained from a pool of convalescent donors which may provide a wider range of anti-viral activity. This diversity is important in overcoming mutations in the virus. Antibody diversity provides a broader range of anti-viral activity by attacking different viral epitopes and enlisting different cellular mechanisms. Neutralization of free virus is mainly the result of steric blocking to prevent infection, whereas additional anti-viral activity may come from activation of effector functions such as complement-mediated or antibody-dependent cellular cytotoxicity.

As the virus continues to mutate, eventually some neutralization of the newer variants will be decreased. As can be seen in Table 3, where four batches of hIVIG manufactured with hybrid plasma have been tested for LVNA. The four batches were manufactured with hybrid plasma collected in early 2021, shortly after the vaccination campaign with original SARS-CoV-2 vaccine which was directed to the ancestral variant. Approximately two and a half years after the collection of the plasma, there is still remarkable neutralization of the SARS-CoV-2 variants.

TABLE 3
Neutralization IC50 titers hyperimmune manufactured
from convalescent plus vaccinated (hybrid) plasma.

| | Variant | | | |
|---|---|---|---|---|
| | WA.01 | BA.5.5 | XBB.1.5 | EG.5.1 |
| | Date of Variant Circulation | | | |
| | January 2020 | | | |
| | (ancestral | mid | February | September |
| | varant) | 2022 | 2023 | 2023 |
| Lot Designation | Neutralization IC50 Titers (1:X) | | | |
| V02 - 1 | 55254 | 16261 | 1096 | 1573 |
| V02 - 2 | 88723 | 14119 | 1160 | 1775 |
| V02 - 3 | 73803 | 15865 | 1283 | 2321 |
| V02 - 4 | 81019 | 16825 | 1578 | 1888 |
| Average | 74700 | 15768 | 1279 | 1889 |

To compensate for lower virus neutralization activity against evolving variants, a larger dose volume may be administered to the patient. Table 4 introduces the concept of dosing units as a measure of neutralization capacity of the hIVIG given to an individual patient for a specified SARS-CoV-2 variant. From Table 4, it is observed that the total neutralization capacity of the V01 hIVIG, as measured by dosing units, decreases with newer variants. Eventually the virus could mutate to such an extent that even with the broadly neutralizing antibodies of the hIVIG, neutralization activity will be reduced. The loss of neutralization activity can be compensated by increasing the volume. It is observed that with subsequent new variants, titers decrease, but with V02 hIVIG, the overall neutralization capacity can be maintained equivalent to the neutralization capacity of V01 hIVIG against the ancestral variant by increasing volume. Thus, V02 can achieve neutralization capacity against new variants at greater than or equal to 344 when administered at volumes less than the 0.300 L needed to achieve the equivalent neutralization capacity by V01 hIVIG against the ancestral variant.

TABLE 4
Variant neutralization capacity of hIVIG product measured in dosing units for V01 and V02
hyperimmune product, manufactured from convalescent plasma and hybrid plasma, respectively.

| | | | Delta | | | | | | | | |
|---|---|---|---|---|---|---|---|---|---|---|---|
| | | | (B.1.617.2, | Omi- | Omi- | Omi- | Omi- | Omi- | Omi- | Omi- | Omi- |
| | | Ances- | circu- | cron | cron | cron | cron | cron | cron | cron | cron |
| | | tral | lating | (BA.1. | (BA.1.1, | (BA.2, | (BA.2.12.1, | (BA.4.1, | (BA.5.5, | (XBB.1.5, | (EG.5.1, |
| Hyperimmune Product | | Variant | August, | January | January | March | June | July | July | February | September |
| and Dosing | | (WA.01) | 2021) | 2022) | 2022) | 2022) | 2022) | 2022) | 2022) | 2023) | 2023) |
| V01 - | V01 | 1147 | 396 | 856 | 239 | 150 | 287 | 297 | n/t² | n/t | n/t |
| Cova- | Average | | | | | | | | | | |
| lescent | Titer | | | | | | | | | | |
| Plasma | (1:X) | | | | | | | | | | |
| | Dose | 0.300 | 0.300 | 0.300 | 0.300 | 0.300 | 0.300 | 0.300 | n/t | n/t | n/t |
| | Volume | | | | | | | | | | |
| | (L) | | | | | | | | | | |
| | V01 | 344 | 119 | 257 | 72 | 45 | 86 | 89 | n/t | n/t | n/t |
| | Dosing¹ | | | | | | | | | | |
| | Units | | | | | | | | | | |
| | (titer * | | | | | | | | | | |
| | Vol, L) | | | | | | | | | | |
| V02 - | V02 | 55254 | n/t | n/t | n/t | n/t | 26792 | 20703 | 15768 | 1279 | 1889 |
| Hybrid | Average | | | | | | | | | | |
| Plasma | Titer | | | | | | | | | | |
| (conval- | (1:X) | | | | | | | | | | |
| escence + | Dose | 0.007 | n/t | n/t | n/t | n/t | 0.013 | 0.017 | 0.022 | 0.270 | 0.182 |
| vaccination) | Volume | | | | | | | | | | |
| | (L) | | | | | | | | | | |
| | V02 | 387 | n/t | n/t | n/t | n/t | 348 | 352 | 347 | 345 | 344 |
| | Dosing | | | | | | | | | | |
| | Units | | | | | | | | | | |
| | (titer * | | | | | | | | | | |
| | Vol, L) | | | | | | | | | | |
| | Ratio to | 1.1 | n/t | n/t | n/t | n/a | 1.0 | 1.0 | 1.0 | 1.0 | 1.0 |
| | Original | | | | | | | | | | |
| | Dosing | | | | | | | | | | |
| | Units | | | | | | | | | | |
| | specific | | | | | | | | | | |
| | to | | | | | | | | | | |
| | ancestral | | | | | | | | | | |
| | variant | | | | | | | | | | |

1-17. (canceled)

18. A pharmaceutical composition comprising human plasma-derived polyclonal, anti-SARS-CoV-2 hyperimmune globulins wherein said plasma is a convalescent plasma from a subject having recovered from SARS-CoV-2 infection and who had been vaccinated from SARS-CoV-2.

19. Pharmaceutical composition according to claim 18, wherein said convalescent plasma is from a subject who has recovered from a non-omicron infection.

20. Pharmaceutical composition according to claim 18, wherein said subject had not been vaccinated from an Omicron SARS-CoV-2 variant.

21. Pharmaceutical composition according to claim 19, wherein said subject had not been vaccinated from an Omicron SARS-CoV-2 variant.

22. Pharmaceutical composition according to claim 18, wherein said composition lack a significant reduction in potency to omicron BA.4, BA.5, XBB and/or EG variants.

23. Pharmaceutical composition according to claim 18, wherein said plasma is a pool of plasma samples from at least two subjects that have been recovered from SARS-CoV-2 infection and who had been vaccinated from SARS-CoV-2.

24. Pharmaceutical composition according to claim 18, wherein said plasma is a pool of plasma samples from at least one hundred subjects that have been recovered from SARS-CoV-2 infection and who had been vaccinated from SARS-CoV-2.

25. A method for treating a SARS-CoV-2 Omicron variant infection comprising administering the pharmaceutical composition according to claim 18.

26. Method according to claim 25, wherein the SARS-CoV-2 Omicron variant infection is SARS-CoV-2 Omicron BA.4 and/or BA.5 variant infection.

27. A method for the preparation of the pharmaceutical composition comprising a polyclonal, hyperimmune globulin composition anti-SARS-CoV-2 according to claim 18, comprising obtaining a plasma sample from a subject having recovered from SARS-CoV-2 infection and who had been vaccinated from SARS-CoV-2.

28. Method according to claim 27, wherein said composition lack a significant reduction in potency to an omicron BA.4, BA.5, XBB and/or EG variants.

29. Method according to claim 27, wherein said plasma sample is a pool of plasma samples from at least two subjects that have been recovered from SARS-CoV-2 infection and who had been vaccinated from SARS-CoV-2.

30. Method according to claim 29, wherein said plasma sample is a pool of plasma samples from at least one hundred subjects that have been recovered from SARS-CoV-2 infection and who had been vaccinated from SARS-CoV-2.

31. A method for obtaining hyperimmune human plasma from a subject for use in the treatment of coronavirus disease 2019 (COVID-19), wherein said subject has a laboratory confirmed diagnosis of COVID-19, is in a convalescent noninfectious state and has been vaccinated from SARS-CoV-2.

32. Method according to claim 31, wherein said subject has been vaccinated from a non-Omicron SARS-CoV-2 variant.

33. A method determining the amount of the pharmaceutical composition comprising human plasma-derived polyclonal, anti-SARS-CoV-2 hyperimmune globulins of the invention to be administered to a subject in need thereof, the method comprising:
a. determining the dominant variant of SARS-CoV-2; and,
b. calculating the amount of the pharmaceutical composition according to the genotype of the dominant variant.

34. The method of claim 33 wherein step (a) comprises measuring the frequency of SARS-CoV-2 variants in a subject to be treated and/or the environmental profile of SARS-CoV-2 variants for that subject.

35. The method of claim 33 wherein step (b) comprises (i) determining the variant neutralization capacity of the pharmaceutical composition for the genotype of the dominant variant, (ii) expressing the neutralization capacity in dosage unit per liter of the pharmaceutical composition, and (iii) calculating the amount of the pharmaceutical composition to be administered to the subject in need thereof.

36. The method of claim 34 wherein step (b) comprises (i) determining the variant neutralization capacity of the pharmaceutical composition for the genotype of the dominant variant, (ii) expressing the neutralization capacity in dosage unit per liter of the pharmaceutical composition, and (iii) calculating the amount of the pharmaceutical composition to be administered to the subject in need thereof.
